(19) United States
(12) Patent Application Publication
(10) Pub. No.: US 2017/0188939 A1
(43) Pub. Date: Jul. 6, 2017

(54) SLEEP MONITORING SYSTEM

(71) Applicant: Huckleberry Labs, Inc., Irvine, CA (US)

(72) Inventors: Jessica Toh, Irvine, CA (US); Seng Oon Toh, Irvine, CA (US)

(21) Appl. No.: 15/399,281

(22) Filed: Jan. 5, 2017

Related U.S. Application Data
(60) Provisional application No. 62/274,912, filed on Jan. 5, 2016.

(51) Int. Cl. A61B 5/00; A61B 5/11

(52) U.S. Cl. CPC A61B 5/4815; A61B 5/0013; A61B 5/0075; A61B 5/0077; A61B 5/7405; A61B 5/1128; A61B 5/486; A61B 5/6892; A61B 2560/0252; A61B 2562/0204; A61B 2562/0233; A61B 2562/029; A61B 2562/222; A61B 2562/227; A61B 2562/0247

(57) ABSTRACT

A sleep monitor system includes a sensor unit, a processing unit and a mobile interface. The sensor unit is arranged to detect a number of variables corresponding to sleep status of a subject. The processing unit is programmed to receive data derived from the number of variables and process the data to produce user output. The mobile interface is arranged to present user output to one or more users.

### TECHNICAL FIELD

The present disclosure relates generally to a sleep monitor system; and more specifically, to a sleep monitor system for indicating sleep and environment related status of a subject to a user.

### BACKGROUND

Sleep disorders are one of the most commonly occurring disorders among a large section of a society. For example, as per a sleep disorder study, approximately 36% of 3-4 year olds wake up every night requiring assistance, and the percentage of younger children requiring assistance is even higher. These disturbances also affect the sleep of other family members. As a result, a large population suffers from chronically disrupted sleep. Further, long-term consequences of chronically disrupted sleep in children include slow growth, chronic irritability, behavioral problems (e.g., aggressiveness, hyperactivity, poor impulse control), poor school performance, family disruption, and maternal depression.

Various solutions exist to address the sleep disruption. According to one known solution, sleep consultants are hired to address the sleep disruption. Generally the sleep consultants are expensive and of varying skill levels. In another known solution, a polysomnography test is conducted to look for underlying sleep disorders. However, the polysomnography test requires an expensive and inconvenient overnight stay at a clinic. In a yet another known solution, a body worn actigraphy unit is used to monitor human rest/activity. However, this solution requires that the subject wear the device. The subject, especially a young child, may not like wearing the device or forget to put it on. Further, various under mattress sensors can be installed to monitor sleeping behavior of the subject. However, these under mattress sensors are riddled with false positive outputs because these sensors do not automatically differentiate between scenarios such as when the subject is present but not moving, and when the subject is not present. In addition, these sensors can be subjected to urine, chewing, accidental laundering, or can cause discomfort.

Therefore, there is a need to provide a method and system for efficiently monitoring sleeping behavior.

### SUMMARY

The present disclosure seeks to provide a sleep monitor system for facilitating recommendations to a user for improving sleep quality of a subject.

In one aspect, a sleep monitor system includes a processing unit, a mobile interface and a sensor unit. The sensor unit further includes a microcontroller, a sensor board having an infrared array sensor and/or a camera module, an ambient light sensor, a microphone, a humidity/temperature sensor and a sensor board ribbon connector. A communications board is operatively coupled with the sensor board through a communications board ribbon connector and further has a WIFI module, a micro SD slot, a micro USB, a speaker, a battery connector, and a battery. A ribbon cable connects the communications board with the sensor board.

Optionally, the sensor unit may include a passive infrared sensor. In addition, the sensor unit may include an ultra-wide band (UWB) radar. More optionally, the sensor unit may include an under mattress pressure sensitive pad wired to the sensor unit.

In another aspect, a sleep monitor system comprises a processing unit, a mobile interface and a sensor unit. The sensor unit further includes a sensor board and a communications board operatively coupled with the sensor board through a communications board ribbon connector and a ribbon cable and a sensor board ribbon connector. A microcontroller is programmed to operate the sensor board and the communications board to record and transmit infrared array sensor data to the processing unit and the mobile interface.

In a yet another aspect, a sleep monitor system comprises a processing unit, a mobile interface and a sensor unit. The sensor unit further includes a sensor board having an infrared array sensor and a communications board operatively coupled with the sensor board through a ribbon cable and further includes a WIFI module. A microcontroller is programmed to operate the infrared array sensor and the WIFI module to record and transmit infrared array sensor data to the processing unit and the mobile interface.

In a yet another aspect, a sleep monitor system comprises a processing unit, a mobile interface and a sensor unit. The sensor unit further includes a sensor board having a camera with an infrared light sources (or alternatively, a plurality of infrared light sources), a communications board operatively coupled with the sensor board through a ribbon cable and further includes a WIFI module. A microcontroller is programmed to operate the camera and the WIFI module to record and transmit images, presence and motion data to the processing unit and the mobile interface.

In a yet another aspect, a sleep monitor system comprises a sensor unit arranged to detect a number of variables corresponding to sleep status of a subject, a processing unit programmed to receive data derived from the number of variables and process the data to produce user output and a mobile interface arranged to present user output to one or more users. Optionally, the sensor unit further comprises a sensor board and a communications board, wherein the communications board is operatively coupled with the sensor board. Further, the sensor board comprises an infrared array sensor, a camera module, a light ambient sensor, a microphone, a passive infra red sensor, a humidity/temperature sensor and a sensor board ribbon connector. Optionally, the communications board may include a WiFi module, a micro SD slot, a micro USB, a speaker, a battery connector, and a battery. A ribbon cable connects the communications board with the sensor board.

In a yet another aspect, a sleep monitor system comprises a sensor unit, a communications board, a microcontroller, a housing surrounding the sensor board and communications board, a processing unit remote from the sensor unit and a mobile interface remote from the sensor unit and the processing unit. The sensor board further includes an infrared array sensor and a passive infrared sensor arranged to detect sleep status of a subject. The communications board is operatively coupled with the sensor board through a ribbon cable and further includes a WIFI module. The microcontroller is programmed to operate the infrared array sensor and the WIFI module to record and transmit data derived from the infrared array sensor and the passive infrared sensor. The housing exposes sensing surfaces of the infrared array sensor and the passive infrared sensor. The processing unit is programmed to receive data derived from the infrared array sensor and passive infrared sensor and process the data to produce user output. A user mobile interface is arranged to present user output to one or more users.

### BRIEF DESCRIPTION OF THE FIGURES

The summary above, as well as the following detailed description of illustrative embodiments, is better understood when read in conjunction with the appended drawings. For the purpose of illustrating the present disclosure, example constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following figures wherein:

FIG. 1 is a schematic illustration of an example sleep monitor system, in accordance with an embodiment of the present disclosure.

FIG. 2 is a schematic illustration of an example sensor unit of the sleep monitor system, in accordance with an embodiment of the present disclosure.

FIG. 3 is a schematic illustration of an example communication board of the sensor unit, in accordance with an embodiment of the present disclosure.

FIGS. 4A and 4B are schematic illustrations of a front perspective view of the sensor board and a rear perspective view of the sensor board respectively, in accordance with an embodiment of the present disclosure.

FIG. 5 is a schematic illustration of a front perspective view of the sensor board having shaded sensor surfaces, in accordance with an embodiment of the present disclosure.

FIG. 6 is a schematic illustration of a perspective view of the sensor unit indicating viewing angles, in accordance with an embodiment of the present disclosure.

### DETAILED DESCRIPTION

The following detailed description illustrates embodiments of the present disclosure and manners by which they can be implemented. Although the best mode of carrying out the present disclosure has been disclosed, those skilled in the art would recognize that other embodiments for carrying out or practicing the present disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

Embodiments of the present disclosure substantially eliminate, or at least partially address, problems in the prior art, and provide a user access to information regarding sleeping behavior of a subject so that the user can improve the sleep quality of the subject by using the sleep monitor system as disclosed herein.

The sleep monitor system of the present disclosure enables the user to monitor sleep of the subject through use of an infrared array sensor and/or a camera (or a combination of both an infrared array sensor and a camera), which can be adapted to track motion as well as presence of the subject during sleep. Further, the sleep monitor system is configured to determine data related to an environment surrounding the subject. The sleep monitor system converts the information collected from the infrared array sensor into sleep data and environment-related information into environment data. The sleep monitor system processes the sleep data and the environment data in order to generate recommendations for the user so that the user can take effective steps to improve the quality of the sleep of the subject. The sleep monitor system can be configured to send the recommendations to the user directly to the communication device as used by the user. Although use of the infrared light source is mentioned above other radiation wavelengths can optionally used for example human eye visible radiation, microwaves, ultrasonic imaging, and others.

The sleep monitor system is designed as a contactless device and the subject is not required to wear the sleep monitor system as generally required in prior art sleep monitor devices. Further, the sleep monitor system is not required to be placed on or under a subject's bed and, as a result, does not provide any discomfort to the subject and is not subjected to bodily fluids or tampering by the subject during night. The sleep monitor system is configured to be operated on a battery and being portable, allows the user to move the sleep monitor system to different positions in order to conform to changing needs of the subject.

The sleep monitor system can be operated in manual or in automatic mode of operation. For example, the user may provide inputs regarding sleep intervals in the manual mode of operation. During the automatic mode of the operation, the sleep monitor system automatically collects the data, for example, sleep and environment data, without requiring any inputs from the user. As a result, accuracy-dependent abilities of the sleep monitor system are significantly increased due to presence of complete and detailed information regarding sleeping habits of the subject. Consequently, the sleep monitor system can generate accurate recommendations to the user on identifying sleep related issues of the subject. For example, in order to forecast a subject's overtiredness (which is a common contributor to disrupted sleep), a complete and accurate set of sleep data is required. Due to availability of complete data, the sleep monitor system can indicate to the user that the subject is facing overtiredness and the user may be required to take appropriate action with the subject in accordance with the level of the overtiredness. The automatic mode of operation makes the sleep monitor system a more robust device, which can be installed and used by the user with own efforts.

Further, a sensing unit of the sleep monitor system is configured to store the sleep data and the environment data within an internal memory and thus, does not rely on a constant internet connection to send the sleep data and environment data to an external processing unit. The sensing unit is configured to internally process the stored data and recommend to the user steps that may be taken for improving seep quality of the subject. Additionally, the sensing unit is configured to transmit the sleep data and the environment data to the external processing unit so that the external processing unit can store the data into a database for further analysis. The sensing unit may synchronize the data stored in the memory with the corresponding data stored in the database. The synchronization can happen automatically whenever the sensing unit discovers an external network providing connectivity to the processing unit.

Further, the processing unit of the sleep monitor system is configured to receive the sleep data and environment data from a plurality of sensing units assigned to a plurality of subjects respectively. As a result, the processing unit becomes a data aggregator for the sleep data of the plurality of subjects and the processing unit is configured to discover trends in the sleeping habits, parameters indicating quality of sleep, sleeping disorders of a category as may be specified by the user. Further, the processing unit can use the data stored in the database to improve recommendations that are provided to the user for a particular subject.

The sleep monitor system and method of recommending the user as disclosed herein can be used for other medical and general health services which may benefit from recommendations based on the sleep data and the environmental data. For example, the recommendations can be used to proactively respond to medical emergencies such as intervention in circumstances where regular movement during the night is required to be monitored so that any health issues can be immediately communicated to the user, on occurrence of seizures during sleep or while determining the effect of medication on sleep of the subject.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

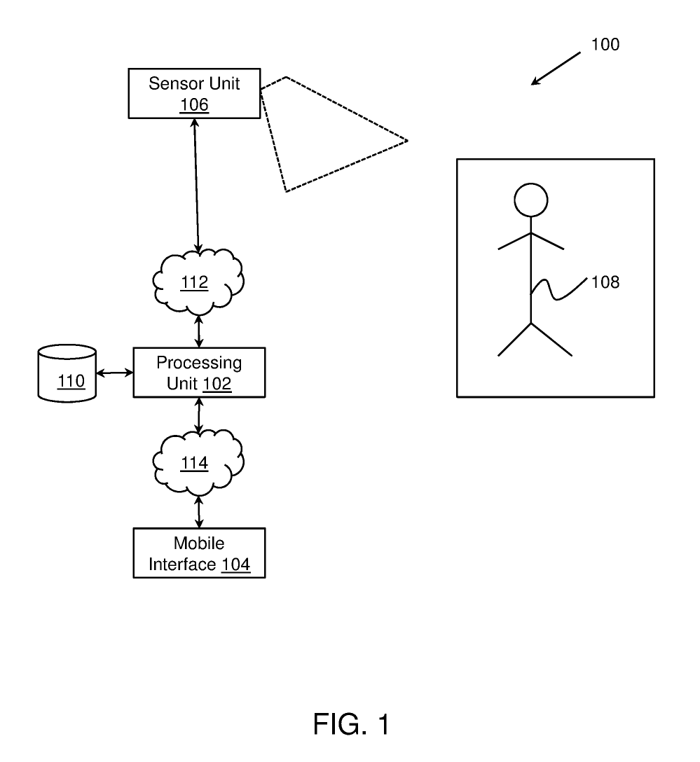

Referring now to the drawings, particularly by their reference numbers, FIG. 1 is a schematic illustration of an example sleep monitor system 100 in accordance with an embodiment of the present disclosure. The sleep monitor system 100 includes a processing unit 102, a mobile interface 104 and a sensor unit 106 configured to communicatively couple to each other. The sensor unit 106 is configured to monitor the sleep data and the environment data for the subject 108 sleeping on a support for example, a bed.

As illustrated in FIG. 1, the sensor unit 106 is communicatively coupled to the processing unit 102 via a communication network 112. The communication network 112 can be a collection of individual networks, interconnected with each other and functioning as a single large network. Such individual networks may be wired, wireless, or a combination thereof. Examples of such individual networks include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, and Worldwide Interoperability for Microwave Access (WiMAX) networks.

The sensor unit 106 transmits information related to the sleep data and the environment data for the subject 108 so that the processing unit 102 can store the information corresponding to the subject 108 in a database 110. The processing unit 102 can include a web server, an analytics server, and a statistics server, which can process the sleep data and the environment data to deliver recommendations to the user. In an embodiment, the database 110 and the processing unit 102 may be implemented in various ways, depending on various possible scenarios. In one example scenario, processing unit 102 and the database 110 may be implemented by way of a spatially collocated arrangement of the processing unit 102 and the database 110. In another example scenario, the processing unit 102 and the database 110 may be implemented by way of a spatially distributed arrangement of the processing unit 102 and the database 110 via a communication network 114. In yet another example scenario, the processing unit 102 and the database 110 may be implemented via cloud computing services.

Further, the processing unit 102 is connected to the mobile interface 104 via a communication network 114 which can be a collection of individual networks, interconnected with each other and functioning as a single large network. Such individual networks may be wired, wireless, or a combination thereof. Examples of such individual networks include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, and Worldwide Interoperability for Microwave Access (WiMAX) networks.

The mobile interface 104 can be an interface selected from an interface of at least one of: smart telephones, Mobile Internet Devices (MIDs), tablet computers, Ultra-Mobile Personal Computers (UMPCs), phablet computers, Personal Digital Assistants (PDAs), web pads, Personal Computers (PCs), handheld PCs, laptop computers, desktop computers, Network-Attached Storage (NAS) devices, large-sized touch screens with embedded PCs, and interactive entertainment devices, such as game consoles, Television (TV) sets and Set-Top Boxes (STBs).

In an embodiment, the user may register the mobile interface 104 with the processing unit 102. Processing unit 102 stores information such as mobile number, a user's identification, an identification of the sensor unit 106 assigned to the user's mobile interface 104 and other related information so as to identify the user. In another embodiment, the user may install application software on the mobile interface 104 so that the application software is configured to directly communicate with the processing unit 102 via the network 114. The application software automatically transfers the information associated with the user to the processing unit 102. The processing unit 102, upon analysis of the sleep data and the environment data associated with the subject 108, transmits recommendations to the mobile interface 104. As a result, the user is able to improve the sleep quality of the subject 108 based on the recommendations.

In another embodiment, the sleep monitor system 100 is configured to provide real time recommendations to the user regarding the sleep quality of the subject 108. During proper network connectivity between the sensor unit 106 and the processing unit 102, the sensor unit 106 is configured to send regular updates of the sleep data and the environment data to the processing unit 102. The processing unit 102 performs the analysis of the received sleep data and the environment data on a real time basis and accordingly, the user will be able to receive recommendations in real time. Otherwise, the sleep monitor system 100 is configured to operate in an offline or intermittent access mode wherein the sensor unit 106 collects and stores the sleep data and the environment data in its internal memory and, upon finding a proper connection with the processing unit 102, transmits the data to the processing unit 102. As a result, the sleep monitor system 100 can provide information to the user on real time basis when a proper connection between the sensor unit 106 and the processing unit 102 is available and in offline mode when no or only intermittent connectivity is available between the sensor unit 106 and the processing unit 102.

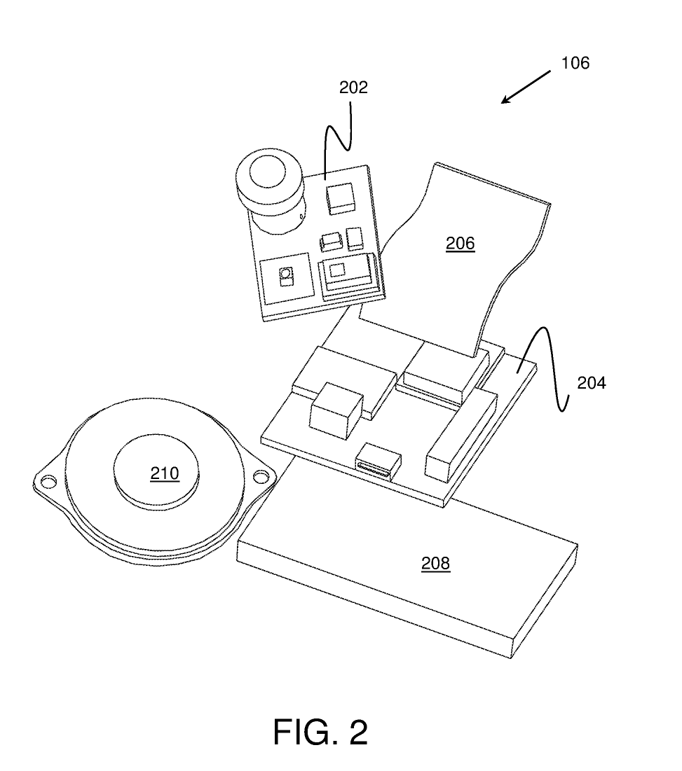

FIG. 2 is a schematic illustration of an example sensor unit 106 in accordance with an embodiment of the present disclosure. The sensor unit 106 includes a sensor board 202, a communication board 204, a ribbon cable 206, a power source 208 and a speaker 210. The sensor board 202 is communicatively coupled to the communication board 204 via the ribbon cable 206. Further, the communication board 204 is connected to the power source 208 so as to provide power to one or more components installed on the communication board 204 and the sensor board 202. The speaker 210 is connected to the power source 208 and a microcontroller (not shown in FIG. 2) in order to generate audio indication to the user regarding the subject 108.

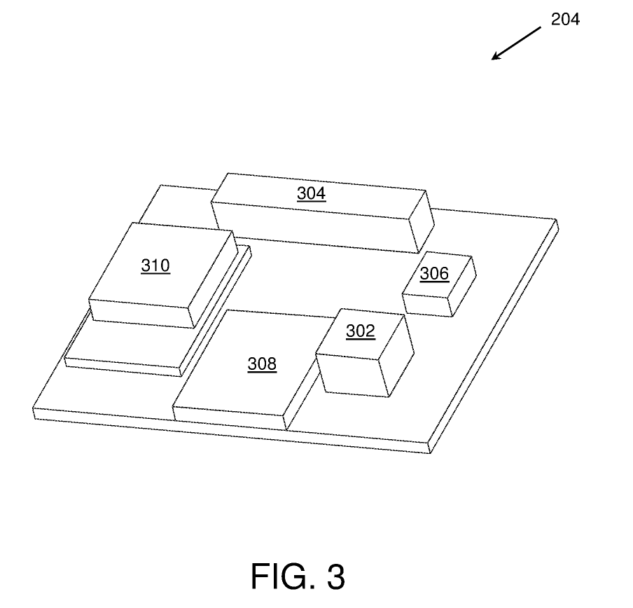

FIG. 3 is a schematic illustration of the communication board 204 in accordance with an embodiment of the present disclosure. The communication board 204 includes a battery connector 302 and a communication board ribbon connector 304. In an embodiment, the communication board 204 is connected to the power source 208 for example, a battery via a pair of power wire so as to receive power from the battery. One end of the pair of wires is connected to the battery connector 302 of the communication board 204 while the other end is connected to another connector in a circuit (not shown) operatively coupled with the battery. Further, the communication board ribbon connector 304 installed on the communication board 204 is used for receiving a first end of the ribbon cable 206 whose other end is being received by a sensor board ribbon connector installed on the sensor board 202. The ribbon cable 206 ensures that the data collected by the various sensors of the sensor board 202 can be transmitted to the communication board 204 so that the communication board 204 can further transmit the sensor data to the processing unit 102.

The communication board 204 further includes a micro universal serial bus (USB) 306, which is used for charging the battery. Thus, the sensor unit 106 can be charged via normal mobile charges, which can be charged through USB ports. The communication board 204 also includes a micro secure digital (SD) card slot 308 for receiving a micro SD card. The micro SD card can be configured to include instructions for securely transmitting the sleep data and the environment data to the processing unit 102. In an embodiment, the micro SD card is installed and fixed while assembling the sensor unit 106 so that the micro SD card remains affixed within the SD card slot 308.

The communication board 204 includes a WIFI module 310 for communicatively coupling to the external devices such as the processing unit 102 or the mobile interface 104. For example, the WIFI module 310 connects with the processing unit 102 via the communication network 112 which may be established by the mobile interface 104.

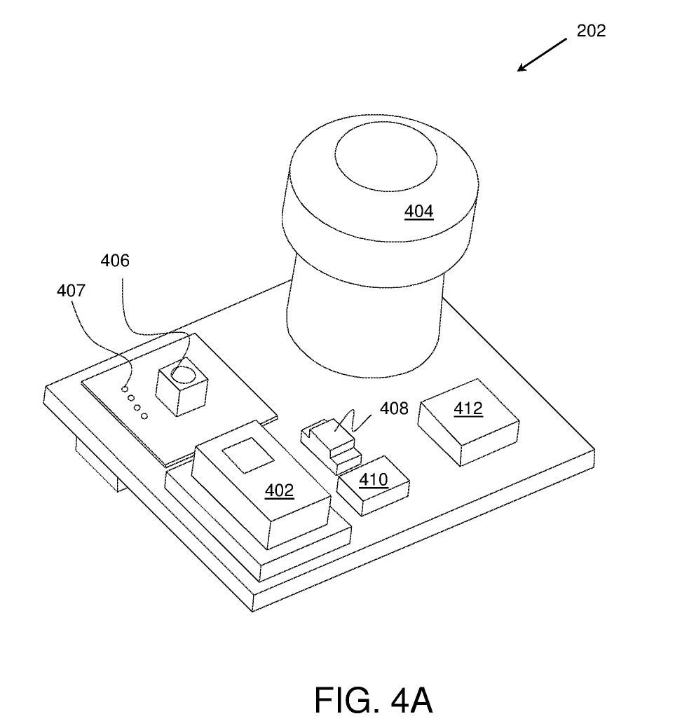

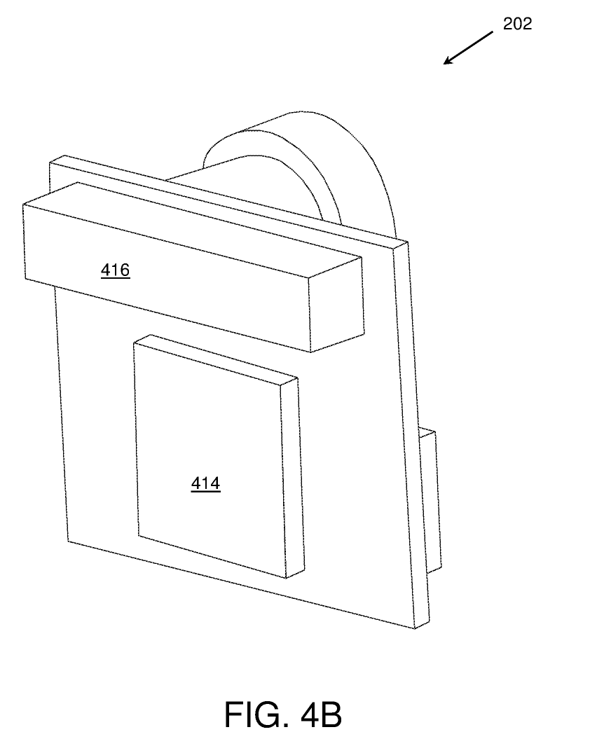

FIG. 4A is a schematic illustration of a front perspective view of the sensor board 202 and FIG. 4B is a schematic illustration of a rear perspective view of the sensor board 202 in accordance with an embodiment of the present disclosure. The front portion of the sensor board 202 includes a plurality of sensors such as an infrared array sensor 402, a passive infrared sensor 404, a camera 406, one or more infrared light sources 407, a light sensor 408, a microphone 410, and a temperature/humidity sensor 412. The rear portion of the sensor board 202 includes a microcontroller 414 and a sensor board ribbon connector 416.

The infrared array sensor 402 and the passive infrared sensor 404 are used to determine presence of the subject 108 within the range of these sensors. Further, these infrared sensors can be used to determine the direction of motion of the subject 108 while sleeping during the night. The camera 406 is used to record or take an image of the subject 108 within a pre-defined range of the camera. In an embodiment, the camera 406 is arranged with a 64-degree solid viewing angle.

In another embodiment, the camera 406 is used to record an image which is then post-processed using image recognition algorithms to determine presence of the subject. A series of sequential images are used to determine the direction of motion of the subject 108 while sleeping during the night.

The light sensor 408 is used to determine light intensity of the ambience so that the output of the light sensor 408 is used as part of the environment data. In an embodiment, a phototransistor is used as the light sensor 408. The microphone 410 is used to determine the level of sound near to the sensor unit 106. The microphone 410 must be sensitive enough to record the sound level within the predefined range as covered by the sensor unit 106. In an embodiment, the microphone 410 is exposed from a case of the sensor unit 106 through a pinhole opening so that the microphone 410 can be adapted to ignore internal noise that may be generated within the casing of the sensor unit 106. The temperature/humidity sensor 412 is used to measure the temperature of the surroundings. In an embodiment, the temperature/humidity sensor 412 is also exposed from the case of the sensor unit 106 so that the temperature/humidity sensor 412 can have accurate samples of the surrounding air to measure temperature and humidity respectively.

Further, the microcontroller 414 of the sensor board 202 is a key component on the sensor unit 106 and is used to control the operations of the sensor board 202 and the communication board 204. Although in FIG. 4B, the microcontroller 414 is mounted on the rear portion of the sensor board 202, the microcontroller 414 can be mounted on the communication board 204. The sensor board 202 is communicatively coupled to the communication board 204 via the ribbon cable 206. Data collected by the plurality of sensors can assist the microcontroller 414 in collecting the sleep data and environment data corresponding to the subject 108. In an embodiment, the microcontroller 414 can be programmed to operate the sensor board 202 and the communications board to record and transmit data from infrared array sensor 402 to the processing unit 102 and the mobile interface 104 or both. In another embodiment, the microcontroller 414 can be programmed to operate the infrared array sensor 402 and the WIFI module 310 to record and transmit infrared array sensor 402 data to the processing unit 102 and the mobile interface 104 respectively.

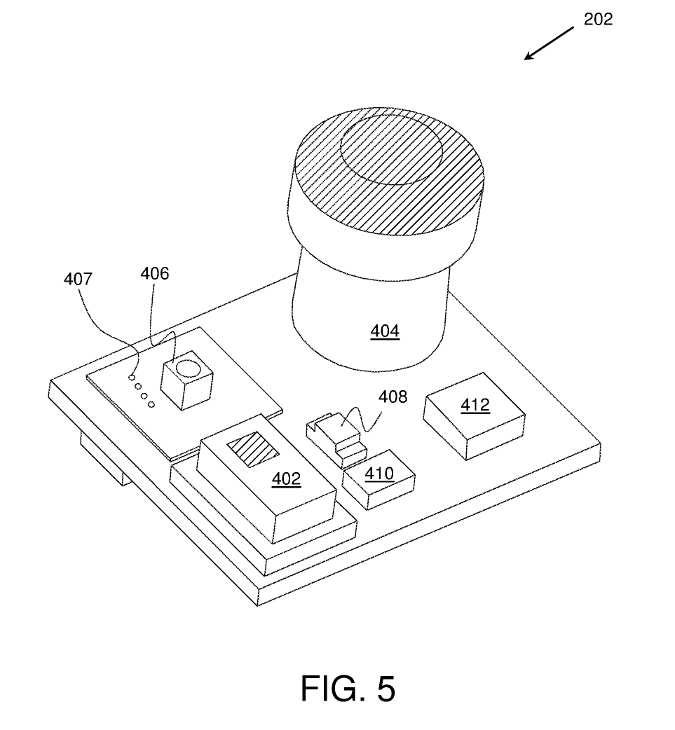

FIG. 5 is a schematic illustration of a front perspective view of the sensor board 202 having shaded sensors surfaces in accordance with an embodiment of the present disclosure. The shaded portions of the infrared array sensor 402 and the passive infrared sensor 404 may remain exposed to the outside of the housing of the sensor unit 106 when the sensor unit 106 is packaged as a single unit in a case which includes the sensor board 202, the communication board 204 and other components of the sensor unit 106. The shaded portions of the infrared array sensor 402 and the passive infrared sensor 404 indicate the respective sensing surfaces.

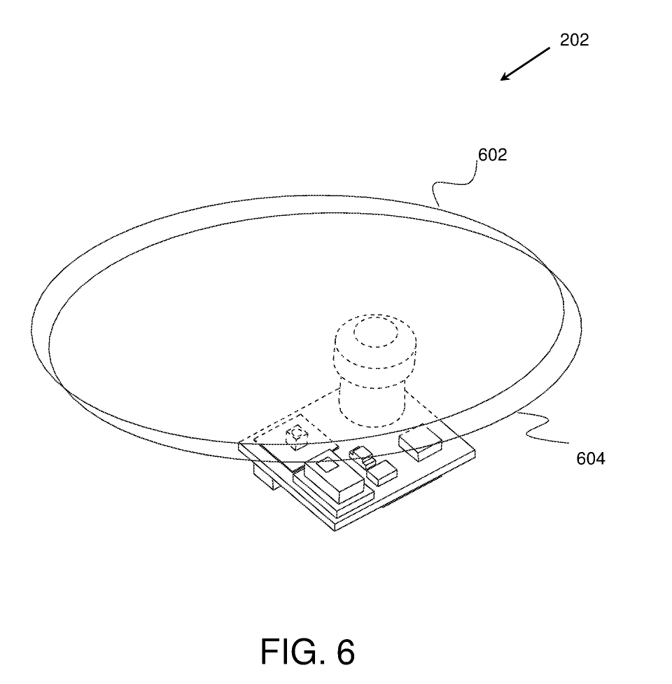

FIG. 6 is a schematic illustration of a perspective view of the sensor unit 106 indicating viewing angles in accordance with an embodiment of the present disclosure. An area within a conical parameter 602 indicates viewing angle of the infrared array sensor 402 and an area within a conical parameter 604 indicates viewing angle of the passive infrared sensor 404. In an embodiment, the infrared array sensor 402 is arranged with a 60 degree viewing angle.

In an optional embodiment, the viewing angle of the camera 406 and infrared sensor 402 are arranged to overlap with each other, for example, to allow observation of the viewpoint of the sensor when using the mobile unit, and consequently, suggest an optimal position for the monitor.

In an embodiment, the sensor unit 106 is configured to detect a number of variables corresponding to the sleep status of the subject 108. For example, the variables can include status of the heater within the range of the subject 108, level of ambient noise, presence of loud noise in the environment, current ambient temperature, level of humidity, ambient light intensity, quality of air, timing of sleep of the subject 108, spacing of the sleep, and other variables that can assist in determining the cause of disruption in the sleep of the subject 108 or determining the quality of the sleep of the subject 108.

As discussed above, the microcontroller 414 is configured to determine the values of these variables using the plurality of sensors present within the sensor board 202 of the sensor unit 106. In an embodiment, the microcontroller 414 is configured to measure the outputs of the infrared array sensor 402 and passive infrared sensor and process these values before transmission to the processing unit 102. Further, the microcontroller 414 is configured to calculate at least one of mean, standard, deviation, natural log, # events>(mean+threshold), and maximum of motion values over centered x - - - minute window values for the motion data as retrieved from the infrared sensors. Subsequently, the microcontroller 414 is configured to determine the presence of the subject 108 or motion of the subject 108 using values of these parameters by comparing with threshold values. In an embodiment, the threshold values are determined using machine learning model. Consequently, the microcontroller 414 derives results or values from the analysis.

In another embodiment, the microcontroller 414 is configured to process the images captured by the camera 406 using image recognition algorithms to determine presence as well as motion of the subject before transmission to the processing unit 102; the image recognition algorithms optionally employ a two dimensional correlation with one or more reference images and/or by making a neural network comparison with an image imprinted on a neural network. In a yet another embodiment, the images captured from the camera 406 are transmitted to the processing unit 102 in a raw or a compressed form. The processing unit 102 is then configured to process these values using the image recognition algorithms to determine presence as well as motion of the subject.

Further, the microcontroller 414 is configured to transmit these values and output of the plurality of sensors to the processing unit 102 using the WIFI module of the communication board 204 of the sleep monitor system 100. Subsequently, the processing unit 102 programmed to receive data derived from the number of variables, processes the data to produce output, which can be further transmitted to the mobile interface 104 and displayed to the user.

Embodiments of the present disclosure are susceptible to being used for various purposes, including, though not limited to, enabling users to receive recommendations for improving sleep quality of the subject through proper analysis of the sleep data and the environment data. Further, embodiments of the present disclosure can be used for receiving alarm signals to proactively respond to any emergency situations through the sleep data and the environment data. The sleep monitor system offers several advantages from the end user perspective. The sleep monitor system as disclosed herein provides a great comfort to the user to operate the device due to presence of an automatic mode of operation. Further, the sleep monitor system can be used for all categories of subjects including but not limited to children, and adults. Furthermore, the sleep monitor system is a contactless system and as a result the sleep monitor system of the present disclosure is not affected by common life occurrences with young children, such as urine accidents, chewing on objects, or ending up in the laundry.

Modifications to embodiments of the present disclosure described in the foregoing are possible without departing from the scope of the present disclosure as defined by the accompanying claims. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A sleep monitor system, comprising:
a processing unit;
a mobile interface; and
a sensor unit further including:
a microcontroller;
a sensor board having an infrared array sensor and/or a camera module with infrared light sources, an ambient light sensor, a microphone, a humidity/temperature sensor and a sensor board ribbon connector;
a communications board operatively coupled with the sensor board through a communications board ribbon connector and further having a WiFi module, a micro SD slot, a micro USB, a speaker, a battery connector, and a battery; and
a ribbon cable connecting the communications board with the sensor board.

2. A sleep monitor system, comprising:
a processing unit;
a mobile interface; and
a sensor unit comprising:
a sensor board including an infrared array sensor;
a communications board operatively coupled with the sensor board through a ribbon cable and including a WiFi module;
a microcontroller programmed to operate the infrared array sensor and the WiFi module to record and transmit infrared array sensor data to the processing unit and the mobile interface.

3. A sleep monitor system, comprising:
a processing unit;
a mobile interface; and
a sensor unit comprising:
a sensor board including a camera with infrared light sources;
a communications board operatively coupled with the sensor board through a ribbon cable and including a WiFi module;
a microcontroller programmed to operate the camera and the WiFi module to record and transmit images, presence, and motion data to the processing unit and the mobile interface infrared array.

4. The sleep monitor system as set forth in claim 3, wherein the sensor board further includes an infrared array sensor, wherein the microcontroller is programmed to operate the infrared array sensor and the WiFi module to record and transmit infrared array sensor data to the processing unit and the mobile interface.

5. The sleep monitor system as set forth in claim 2, wherein:
the sensor unit is arranged to detect a number of variables corresponding to sleep status of a subject;
the processing unit is programmed to receive data derived from the number of variables and process the data to produce user output; and
the mobile interface is arranged to present user output to one or more users.

6. The sleep monitor system as set forth in claim 5, wherein the microcontroller is programmed to operate the sensor board and the communications board to record and transmit data derived from the number of variables to the processing unit, the mobile interface or both.

7. The sleep monitor as set forth in claim 2, wherein the sensor board further includes a camera module with infrared light sources, wherein the infrared array sensor and the camera module are arranged with overlapping viewing angles and the microcontroller is programmed to operate the infrared array sensor, the camera module and the WiFi module to record and transmit images, presence, and motion data to the processing unit and the mobile interface.

8. The sleep monitor system as set forth in claim 7, wherein the infrared array sensor is arranged with a 60 degree viewing angle and the camera module is arranged with a 64-degree solid viewing angle.

9. The sleep monitor system as set forth in claim 2, wherein the sensor board further comprises an ambient light sensor.

10. The sleep monitor system as set forth in claim 2, wherein the sensor board further comprises a microphone.

11. The sleep monitor system as set forth in claim 2, wherein the sensor board further comprises a passive infrared sensor.

12. The sleep monitor system as set forth in claim 2, wherein the sensor board further comprises a humidity/temperature sensor.

13. The sleep monitor system as set forth in claim 2, wherein the sensor board further comprises a sensor board ribbon connector, the communications board further comprises a communications board ribbon connector and the ribbon cable coupling the communications board ribbon connector with the sensor board ribbon connector.

14. The sleep monitor system as set forth in claim 2, wherein the communications board further comprises a micro SD slot.

15. The sleep monitor system as set forth in claim 2, wherein the communications board further comprises a micro USB.

16. The sleep monitor system as set forth in claim 2, wherein the communications board further comprises a battery connector.

17. The sleep monitor system as set forth in claim 2, wherein the communications board further comprises a speaker.

18. The sleep monitor system as set forth in claim 2, wherein the communications board further comprises a power source.

19. The sleep monitor system as set forth in claim 17, wherein the power source further comprises a battery.

20. The sleep monitor system as set forth in claim 2, wherein the microcontroller is mounted to the sensor board or the communications board.
